(19) United States
(12) Patent Application Publication
(10) Pub. No.: US 2006/0069369 A1
(43) Pub. Date: Mar. 30, 2006

(54) Absorbent system design for absorbent articles

(75) Inventors: Joseph R. Feldkamp, Appleton, WI (US); Richard B. Timmers, Appleton, WI (US); Richard N. Dodge II, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/231,246

(22) Filed: Sep. 19, 2005

Related U.S. Application Data
(60) Provisional application No. 60/615,161, filed on Sep. 30, 2004.

(51) Int. Cl. A61F 13/15

(52) U.S. Cl. 604/378

(57) ABSTRACT

The present invention relates to disposable absorbent articles that include absorbent systems. The absorbent system includes an absorbent pad formed of absorbent materials and having a lateral direction and a longitudinal direction. The absorbent pad has a narrow zone area of increased thickness of absorbent materials that has a length greater than or equal to 80% of the length of the absorbent pad. The narrow zone area has a width in the lateral direction of the absorbent pad that is less than or equal to 62% and greater than or equal to 20% of the width of a non-zone area.

This application claims priority to provisional application Ser. No. 60/615,161 entitled Improved Absorbent System Design For Absorbent Articles and filed in the U.S. patent and Trademark Office on Sep. 30, 2004. The entirety of provisional application Ser. No. 60/615,161 is hereby incorporated by reference.

### BACKGROUND OF THE INVENTION

Absorbent articles, particularly disposable absorbent articles, have undergone an evolution since they first became commercially available. Materials, features and designs made available by new technologies have improved the function and fit of absorbent articles. An important functional feature of absorbent articles is the ability to intake and contain bodily wastes such as urine and feces. Reliably successful intake and containment of urine can be challenging when the absorbent article is worn by an individual that spends significant time in a prone position, such as an infant or an adult who has lost mobility.

Significant research and technology efforts have been dedicated to designing absorbent systems for absorbent articles that are capable of meeting consumers' expectations for zero leakage regardless of the age or body shape of the wearer. Absorbent systems for commercially available absorbent articles typically include multiple layers. For example, an absorbent system may include a surge layer (to assist with intake) and an absorbent pad that is a mixture of superabsorbent material and an absorbent filler material, such as cellulose fluff. Improved intake times can be achieved by increasing the thickness of the absorbent pad in the intake region of an absorbent article. For a disposable diaper, the intake region may be the crotch region which is the portion of the diaper that extends between the wearer's legs. A potential downside of designing the absorbent system to have an absorbent pad that is thicker in the intake region is that fluid does not distribute throughout the entire absorbent pad as well and consequently, the absorbent pad becomes very bulky, particularly in the crotch region, when it is wet. In addition to making the absorbent article less comfortable, the increased bulk may interfere with the article's seals around the leg openings and may result in increased leakage. Alternatively, the multiple layer absorbent system may be designed to have a very thin and dense absorbent pad. While a very thin, but dense absorbent pad may provide benefits such as improved fluid distribution, comfort and fit, the intake time is typically unacceptable. Consequently, there remains a need for an absorbent system that can be used in a disposable absorbent article that has good fluid distribution after multiple insults, keeps fluid away from the edges of the absorbent to minimize leaks and maintains acceptable intake times.

### SUMMARY OF THE INVENTION

The present invention is directed to a disposable absorbent article including an absorbent system. The absorbent system may include an absorbent pad formed of absorbent materials and having a lateral direction and a longitudinal direction. The absorbent pad may have a narrow zone area of increased thickness of absorbent materials. The narrow zone area has a thickness that is greater than the thickness of-a non-zone area of the absorbent pad. The narrow zone area may have a width in the lateral direction of the absorbent pad that is less than or equal to 62% and greater than or equal to 20% of the width of the non-zone area of the absorbent pad. The narrow zone area may also have a length in the longitudinal direction of the absorbent pad that is greater than or equal to 80% of the length of the absorbent pad.

The narrow zone area may have a thickness that is 1.2 to 5 times the thickness of the non-zone area. The narrow zone area may have a thickness that is at least 0.1 centimeters greater than the thickness of the non-zone area. Alternatively, the narrow zone area may have a thickness that is at least 0.4 centimeters greater than the thickness of the non-zone area. The absorbent materials in the narrow zone area may have a density that is the same as the density of the absorbent materials in the non-zone area of the absorbent pad. Alternatively, the absorbent materials in the narrow zone area may have a density that is less than the density of the absorbent materials in the non-zone area of the absorbent pad. The absorbent system of the disposable absorbent article may also include a surge layer. Typically, the surge layer is positioned between the liner and the absorbent pad of a disposable absorbent article. The absorbent pad, including both the narrow zone area and the non-zone area, may be a continuous structure or it may be formed of two or more layers. For example, with a two-layer absorbent pad, the narrow zone area may be one layer and the non-zone area may be the other layer. With such a system, the narrow zone area layer and the non-zone area layer would be adjacent to each other or in very close proximity to each other.

### BRIEF DESCRIPTION OF THE DRAWINGS

The present invention will be more fully understood and further advantages will become apparent when reference is made to the following detailed description of the invention and the accompanying drawings wherein like numerals represent like elements.

The drawings are merely representative and are not intended to limit the scope of the appended claims.

FIG. 1 representatively shows a planar view of an absorbent system having a conventional zoning of absorbent material that extends across the full width of the absorbent pad;

FIG. 2 representatively shows a planar view of an absorbent system of the invention having a narrow zone of an increased thickness of absorbent material that extends the full length of the absorbent pad;

FIG. 3 representatively shows a perspective view of a three-dimensional finite element mesh used to perform computer simulations on various absorbent system designs;

FIG. 4 representatively shows a computer-generated fluid distribution contour map for an absorbent system having a conventional zoning of absorbent material;

FIG. 5 representatively shows a computer-generated fluid distribution contour map for an absorbent system of the present invention having a narrow zone of increased thickness of absorbent material that has a length substantially the same as the length of the absorbent pad;

FIG. 6 representatively shows a computer-generated fluid distribution contour map from a second series of simulations for an absorbent system having a conventional zoning of absorbent materials;

FIG. 7 representatively shows a computer-generated fluid distribution contour map from a second series of simulations for an absorbent system of the present invention;

FIG. 8 representatively shows a computer-generated fluid distribution contour map from a second series of simulations for an absorbent system having a conventional zoning of absorbent materials;

FIG. 9 representatively shows a computer-generated fluid distribution contour map from a second series of simulations for an absorbent system having a conventional zoning of absorbent materials in a shaped absorbent pad;

FIG. 10 representatively shows a computer-generated fluid distribution contour map from a second series of simulations for an absorbent system of the present invention including a shaped absorbent pad;

FIG. 11 representatively shows a graphical depiction of Liquid Distribution (units of grams/square centimeters) versus Distance Along the Longitudinal Center Axis of the Absorbent Pad (units of centimeters) based on the second series of simulations; the graph depicts the effect of the zoning of absorbent materials on the distribution of liquid within an absorbent pad;

FIG. 12 representatively shows a planar view of a typical disposable absorbent article in which the absorbent systems of the present invention may be incorporated;

FIG. 13 representatively shows a cross-section of an absorbent system of the invention including an absorbent pad and showing the difference in the thickness of the narrow zone area and the non-zone area; and

FIG. 14 representatively shows a cross-section of another absorbent system of the invention including an absorbent pad and showing the difference in the thickness of the narrow zone area and the non-zone area.

### DETAILED DESCRIPTION OF THE INVENTION

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is directed to solving problems related to providing absorbent systems for disposable absorbent articles that have improved fluid distribution, reduced leakage and maintain good intake times. Desirably, absorbent systems function to receive multiple fluid insults in such a way that the fluid is distributed uniformly along the length of an absorbent pad and the fluid is not concentrated along the longitudinal edges of the absorbent pad (in order to reduce leakage).

The present invention relates to an improved absorbent system design. The improved design includes several aspects. For example, absorbent systems of the present invention include an absorbent pad that has increased thickness of absorbent materials (both superabsorbent material and absorbent filler) within a narrow zone area that extends along the majority of the length of the absorbent pad. The increased thickness may be achieved with higher basis weight, lower density or other configurations of absorbent materials. Absorbent articles typically have a longitudinal direction that coincides with the length of the article and a lateral direction that coincides with the width of the article. The different regions of a disposable diaper are typically a front waist region, a crotch region and a back waist region where the length of the article is measured from the lateral edge of the front waist region to the lateral edge of the back waist region. The width of the article is measured from one longitudinal edge to the other longitudinal edge and typically varies for the different regions of the article. For example, typically the width in the crotch region is the narrowest. The absorbent pad portion of the absorbent system may extend the full length of the absorbent article or it may extend over a portion of the length. With the absorbent systems of the invention, the narrow zone area of increased thickness has a width that is significantly less than the width of the non-zone area in the crotch region. The absorbent systems of the invention have improved fluid distribution along the length of the absorbent pad without compromised intake times and result in improved comfort and fit of the article. The improvements are believed to result because the absorbent pad remains relatively thin, even when wet, along the lateral edges of the article.

Typically, thicker absorbent pads have better intake rates than thin absorbent pads. An explanation for this result is that liquid transport becomes boundary-limited in the Z-direction (thickness) before it becomes limited in the Y-direction (length) or X-direction (width). However, there seems to be a “critical thickness” that is the maximum thickness for which an intake benefit can be achieved. Thicknesses greater than the “critical thickness” do not result in additional intake improvement. The “critical thickness” can be calculated, using the following equation, if the moisture diffusivity, D, is known:

$${Z}_{crit} = \sqrt{\frac{Dt}{n}}$$

In this equation, “n” represents porosity of the absorbent pad and “t” represents time. For an airlaid absorbent pad, a conservative estimate of moisture diffusivity is 0.2 cm²/sec for an absorbent pad having a porosity, n, of 0.8 and a typical insult time of 5 seconds. Applying these values to the equation above, a thickness of about 1.0 cm could be effectively wetted (interstitial saturation>=0.5) in the time frame of an insult. A thickness of about 0.5 cm would wet out in a time frame of 1.2 seconds. These thicknesses are greater than what is typically used in absorbent articles, therefore intake performance is not maximized. Increased absorbent pad thickness also aids in-plane liquid transport because there is greater cross-sectional area available for flow in the longitudinal and lateral directions.

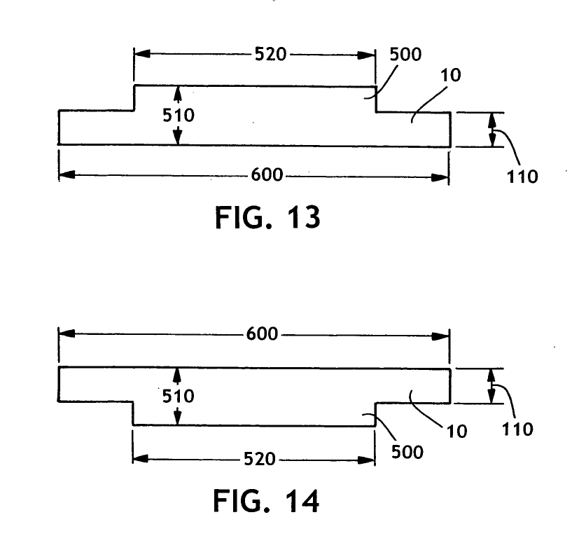

The absorbent systems of the present invention include an absorbent pad that has a narrow zone area of increased thickness of absorbent materials positioned along, and extending laterally outward, from the lateral centerline (which can also be referred to as the centerline running in the longitudinal direction) of the article. The narrow zone area of increased thickness may be located above or beneath a relatively thin main absorbent layer of the absorbent pad (See FIGS. 13 and 14). The narrow zone area may have a length in the longitudinal direction of the article that is substantially the same as the length of the absorbent pad. The narrow zone area may also have a length in the longitudinal direction of the article that is greater than or equal to ninety-five percent of the length of the absorbent pad. Alternatively, the narrow zone area may have a length in the longitudinal direction of the article that is greater than or equal to ninety percent of the length of the absorbent pad. The narrow zone area may also have a length in the longitudinal direction of the article that is greater than or equal to eighty percent of the length of the absorbent pad. The narrow zone area may have a width in the lateral direction of the article that is less than or equal to sixty-two percent of the width of the non-zone area of the absorbent pad. Alternatively, the narrow zone area may have a width in the lateral direction of the article that is less than or equal to sixty percent of the width of the non-zone area of the absorbent pad. The narrow zone area may also have a width in the lateral direction of the article that is less than or equal to fifty-five percent of the width of the non-zone area of the absorbent pad. Additionally, the narrow zone area may have a width in the lateral direction of the article that is less than or equal to fifty percent of the width of the non-zone area of the absorbent pad. The narrow zone area may have a width in the lateral direction of the article that is less than or equal to forty-five percent of the width of the non-zone area of the absorbent pad. The narrow zone area may also have a width in the lateral direction of the article that is less than or equal to forty percent of the width of the non-zone area of the absorbent pad. Further, the narrow zone area may have a width in the lateral direction of the article that is less than or equal to thirty percent of the width of the non-zone area of the absorbent pad. The narrow zone area may also have a width in the lateral direction of the article that is greater than or equal to twenty percent of the width of the non-zone area of the absorbent pad. The design of the absorbent systems of the invention improves the flow of fluid along the longitudinal direction of the absorbent pad and therefore, fluid is more likely to be kept away from the longitudinal edges of the article and as a result, have less contact with the wearer's skin. Therefore, the absorbent systems of the invention are believed to provide skin health benefits in addition to improved fit, comfort and leakage control.

The absorbent pad may be formed of a continuous structure of absorbent materials or it may be formed of two or more layers of absorbent materials that are adjacent to each other. With absorbent pads having a continuous structure of absorbent materials, the narrow zone area and the non-zone area are continuous with each other and the narrow zone area has a thickness that is in addition to the thickness of the non-zone area. Therefore, the narrow zone area has an increased thickness of absorbent materials compared to the non-zone area. With absorbent pads having two or more adjacent layers of absorbent materials, the narrow zone area may be a layer of absorbent materials and non-zone area may be a separate layer of absorbent layers. The thickness of the narrow zone area is the sum of the thicknesses of both layers and therefore, the narrow zone area has an increased thickness of absorbent materials compared to the non-zone area.

Description of a Typical Disposable Absorbent Article in which the Absorbent Systems of the Invention May Be Used

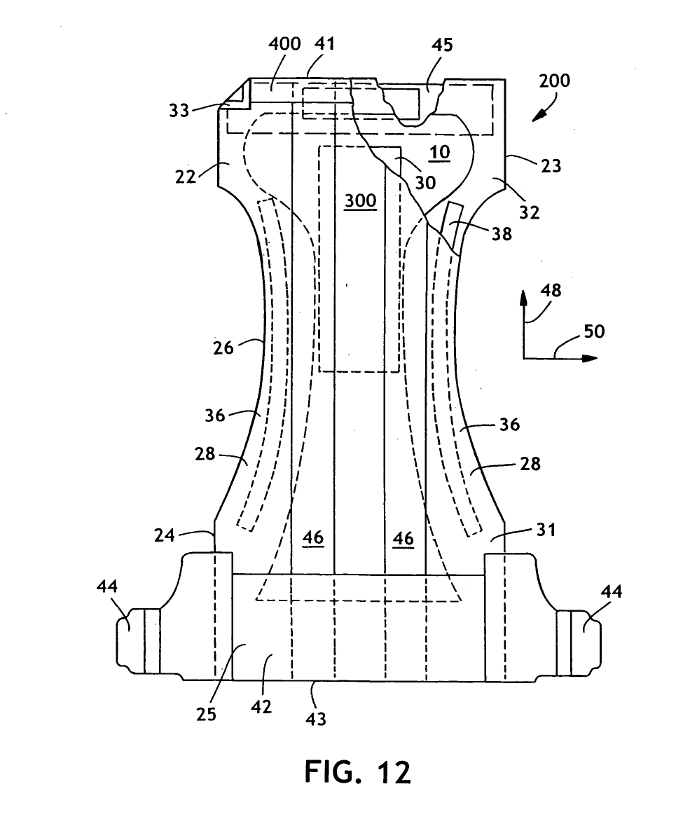

FIG. 12 representatively illustrates an embodiment of a disposable absorbent article 200 in which the absorbent systems of the present invention may be used. The surface of the article which contacts the wearer is facing the viewer. The absorbent article 200 defines a front portion 22, a rear portion 24 and a crotch portion 26 connecting the front portion 22 and the rear portion 24. The front portion 22 defines a front waist region 23 and includes a front waist edge 41. The rear portion 24 defines a rear waist region 25 and includes a rear waist edge 43. The absorbent article 200 also defines a longitudinal direction 48 and a lateral direction 50. The absorbent article 200 includes a bodyside liner 300, an outer cover 32 and an absorbent pad 10 located between the bodyside liner 300 and the outer cover 32. The absorbent article 200 includes an inner surface 31 generally oriented towards the skin of the wearer and an outer surface 33 generally oriented towards the clothing of the wearer.

As used herein, reference to a front portion refers to that part of the absorbent article which is generally located on the front of a wearer when in use. Reference to a front waist region refers to that part of the front portion which is located generally near the waist opening. Reference to the rear portion refers to the portion of the article generally located at the rear of the wearer when in use. Reference to a rear waist region refers to that part of the rear portion which is located generally near the waist opening. Reference to the crotch portion refers to that portion which is generally located between the legs of the wearer when in use. In some embodiments, the liner forms the inner surface. In some embodiments, the outer cover forms the outer surface.

The crotch portion 26 has opposite longitudinal side portions 28 which may include a pair of elasticized, longitudinally-extending leg cuffs 36. The leg cuffs 36 are generally adapted to fit about the legs of a wearer in use and serve as a mechanical barrier to the lateral flow of body exudates. The leg cuffs 36 may be elasticized by a pair of leg elastics 38. The absorbent article 200 may further include a front waist elastic 400 and/or a rear waist elastic 42. The rear portion 24 of the absorbent article 200 may further include a fastening means 44 which is intended to hold the absorbent article 200 about the waist of the wearer when in use. The absorbent article 200 may also include a pair of containment flaps 46 which extend longitudinally along the absorbent article 200 and are also adapted to provide a barrier to the flow of body exudates. It should be recognized that individual components of the absorbent article 200, such as the elastic members, may be optional depending upon the intended use of the absorbent article 200.

The bodyside liner 300 of the absorbent article 200 suitably presents a bodyfacing surface which is intended to be worn adjacent the body of the wearer and is compliant, soft feeling and nonirritating to the wearer's skin. A suitable bodyside liner 300 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

The outer cover 32 of the absorbent article 200 may suitably be composed of a material which is either liquid permeable or liquid impermeable. It is generally preferred that the outer cover 32 be formed from a material which is substantially impermeable to fluids. For example, a typical outer cover can be manufactured from a thin plastic film or other flexible liquid-impermeable material. For example, the outer cover 32 may be formed from a polyethylene film. If it is desired to present the outer cover 32 with a more clothlike feeling, the outer cover 32 may comprise a polyethylene film having a nonwoven web laminated to the outer surface thereof, such as a spunbond web of polyolefin fibers. Methods of forming such clothlike outer covers are known to those skilled in the art.

The bodyside liner 300 and outer cover 32 are generally joined together so as to form a pocket in which the composite absorbent pad 10 is located. The bodyside liner 300 and outer cover 32 may be joined directly to each other around the outer periphery of the absorbent article 200 by any means known to those skilled in the art such as, for example, adhesive bonds, sonic bonds or thermal bonds. Such bonding means may also be suitable for attaching other components of the composite absorbent pad and absorbent article of the present invention together. The leg cuffs 36 are suitably formed by portions of the outer cover 32, and/or bodyside liner 300, which extend beyond the longitudinal sides of the composite absorbent pad 10. Naturally, the leg cuffs 36 may also be formed from separate materials which are attached to the outer cover 32 and/or bodyside liner 300.

The leg cuffs 36 may include leg elastics 38. Waist elastics 400 and 42 may also be provided. The leg elastics 38 are arranged to draw and hold the absorbent article 200 against the legs of the wearer. The waist elastics 400 and 42 are also arranged to draw and hold the absorbent article 200 against the wearer. Materials suitable for use in forming leg elastics 38 and waist elastics 400 and 42 are known to those skilled in the art. Exemplary of such materials are strands or ribbons of a polymeric, elastomeric material which are adhered to the absorbent article 200 in a stretched position, or which are attached to the absorbent article while the article is pleated, such that elastic constrictive forces are imparted to the absorbent article 200. In a particular aspect of the invention, the elastics may be composed of individual strands of LYCRA which are available from INVISTA Co., a business having offices in Wichita, Kans., USA.

The leg elastics 38 and waist elastics 400 and 42 may have any configuration which provides the desired performance. For example, the leg elastics 38 and waist elastics 400 and 42 may comprise a single strand of elastic material, or may comprise several parallel or non-parallel strands of elastic material. The leg elastics 38 may be generally straight or optionally curved to more closely fit the contours of the legs and buttocks of the wearer and better contain bodily exudates. The leg elastics 38 and waist elastics 400 and 42 may be attached to the absorbent article 200 in any of several ways which are well known to those skilled in the art. For example, the elastics may be ultrasonically bonded, thermally bonded or adhesively bonded to the absorbent article 200.

The fastening means 44 are typically applied to the corners of the rear portion 24 of the absorbent article 200 to provide a means for holding the article 200 on the wearer. Suitable fastening means 44 are well known to those skilled in the art and can include tape tab fasteners, hook and loop fasteners, mushroom and loop fasteners, snaps, pins, belts, and the like, and combinations thereof. Typically, the fastening means 44 are configured to be refastenable. It should also be understood that it may be possible to dispense with the fastening means 44 in an absorbent article having a given design configuration.

In some embodiments, the fastening means 44 may be adapted to engage or otherwise join with a fastener landing material 45. Typically, the fastener landing material 45 is a loop material joined to the outercover 32 in the front waist region 23 and is adapted to engage hook-type fastening means 44. In alternative embodiments, the fastener landing material 45 may be a film adapted to engage with tape tab fastening means 44.

The absorbent pad 10 is positioned between the bodyside liner 300 and the outer cover 32 to form the absorbent article 200. The absorbent pad 10 is generally conformable and capable of absorbing and retaining body exudates. It should be understood that, for the purpose of the present invention, the absorbent pad 10 may comprise a single, integral piece of material or, alternatively, may comprise a plurality of individual separate pieces of material which are operably assembled together.

The absorbent pad 10 may have any of a number of shapes and sizes. The absorbent pad 10 may suitably comprise various types of wettable, hydrophilic fibrous materials. Examples of suitable materials include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester and polyamide fibers; and synthetic fibers composed of a nonwettable thermoplastic polymer, such as polypropylene fibers, which have been hydrophilized by appropriate means known to those skilled in the art. The absorbent pad 10 may also comprise selected blends of the various types of fibers mentioned above. The absorbent pad 10 may include a matrix of hydrophilic fibers, such as a web of cellulosic fibers, mixed with particles of a high-absorbency material such as that commonly known as superabsorbent material.

The absorbent article 200 of the present invention may also contain a surge layer 30 to advantageously improve the overall fluid intake rate of the absorbent pad 10. The surge layer 30 is typically less hydrophilic than the absorbent pad 10 and is configured to collect and temporarily hold fluid surges. This configuration can also help prevent fluid exudates from pooling and collecting on portions of the absorbent pad 10.

### PERFORMANCE OF ABSORBENT SYSTEMS OF THE INVENTION

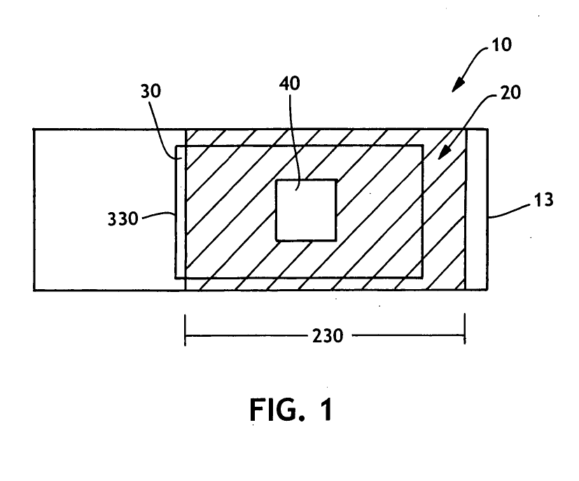

A series of computer simulations using finite element analysis was run to compare the fluid intake and fluid distribution of absorbent systems having a design of the present invention with designs of known absorbent systems. The “known” absorbent system design included a “standard” zoned absorbent. The general configuration of the standard zoned absorbent representing a known absorbent system is representatively illustrated in FIG. 1. The absorbent pad 10 includes a region of zoned absorbent material 20 (the region 20 is represented in FIG. 1 by cross-hatching) that extends from just ahead of the back edge 330 of a surge layer 30 to almost the front edge 13 of the absorbent pad 10. The length of the region of zoned absorbent material is indicated by reference numeral 230. The predicted insult zone 40 for the absorbent pad 10 is representatively shown in FIG. 1. The length of the region of zoned absorbent material 230 is approximately 64% of the total length of the absorbent pad 10 and the width is the entire width of the absorbent pad 10. The region of zoned absorbent material 20 may have a thickness that is greater than the thickness of the remainder of the absorbent pad 10. In the simulation, the region of zoned absorbent material 20 had a thickness of 0.46 cm and the remainder of the absorbent pad 10 had a thickness of 0.30 cm. Thus, the region of zoned absorbent material 20 was 0.16 cm thicker than the rest of the absorbent pad 10.

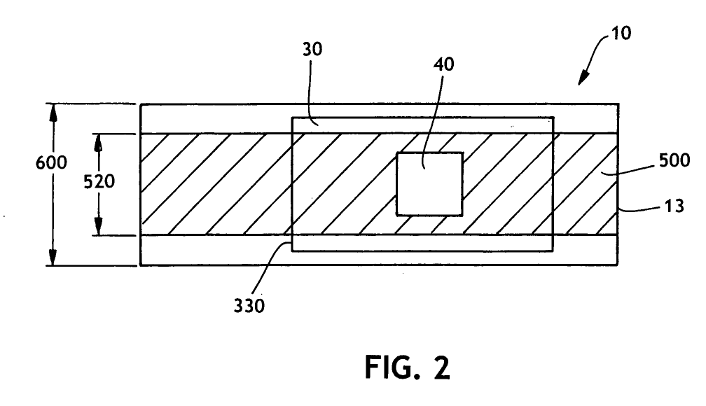

The design of the absorbent system of the invention used for the simulation included a narrow zone area of increased thickness of absorbent materials having a length extending the full length of the absorbent pad in the longitudinal direction and having a width representing 62% of the width of the non-zone area of the absorbent pad in the lateral direction of the article. In the simulation, the narrow zone area of increased thickness had a thickness of 0.46 cm and the non-zone area of the absorbent pad had a thickness of 0.30 cm. Therefore, the narrow zone area was 0.16 cm thicker than the non-zone area of the absorbent pad. A general configuration of an absorbent pad including a narrow zone area representing an absorbent system of the invention is representatively illustrated in FIG. 2. The design of the absorbent pad 10 of the invention includes a narrow zone area of absorbent material 500 that extends the full length of the absorbent pad 10 in the longitudinal direction of an article. The narrow zone area of absorbent material 500 has a width 520 that is representatively illustrated in FIG. 2 as 62% of the width 600 of the non-zone area of the absorbent pad 10. The absorbent system representatively illustrated in FIG. 2 also includes a surge layer 30 and a representation of the insult zone 40. For both absorbent system designs tested through the simulation, the capacity of the absorbent pads was the same, about 550 grams of saline. FIGS. 13 and 14 also representatively illustrate absorbent pads 10 that may be incorporated into absorbent systems of the invention. FIG. 13 representatively shows a cross-section of an absorbent pad 10 having a narrow zone area 500 that may be oriented toward the inner surface 31 of a disposable absorbent article 200. The narrow zone area 500 has a thickness 510 that is greater than the thickness 110 of the non-zone area of the absorbent pad 10. The narrow zone area 500 also has a width 520 that is representatively shown in FIG. 13 to be 62% of the width 600 of the non-zone area of the absorbent pad 10. FIG. 14 representatively shows a cross-section of an absorbent pad 10 having a narrow zone area 500 that may be oriented toward the outer surface 33 of a disposable absorbent article 200. The narrow zone area 500 has a thickness 510 that is greater than the thickness 110 of the non-zone area of the absorbent pad 10. The narrow zone area 500 also has a width 520 that is representatively shown in FIG. 14 to be 62% of the width 600 of the non-zone area of the absorbent pad 10.

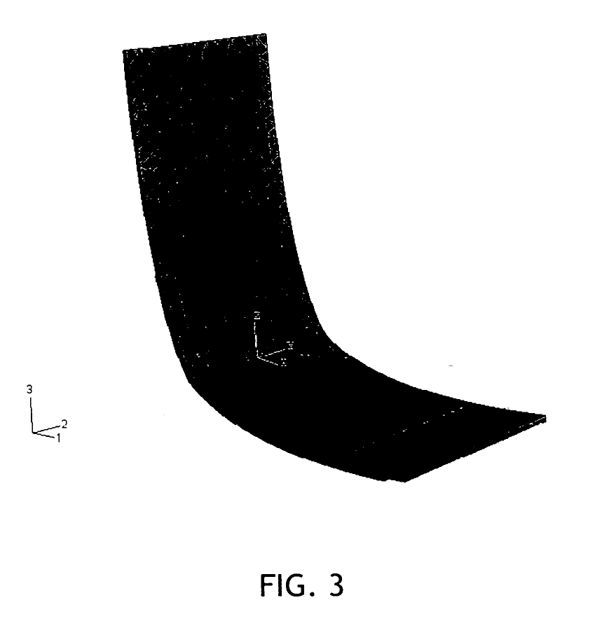

The first set of computer simulations were run in a way to simulate the geometry that the absorbent systems would have if they were part of an absorbent article being worn by a person in the prone position. The simulated geometry (three-dimensional finite element mesh) is representatively illustrated in FIG. 3. The sequence of the simulation included the following events: (1) a 70 gram liquid insult; (2) a 60 second hold time (in which no liquid is allowed to enter the absorbent system, however liquid may re-distribute itself within the absorbent system); (3) a second, 70 gram liquid insult; and (4) a second, 60 second hold time. The simulation included predictions of intake time and liquid distribution after completion of all of the simulation events. In addition to the absorbent pad 10 configurations shown in FIG. 1 (“known” absorbent system) and FIG. 2 (absorbent system of the invention), the simulations also included the additional components of a spunbond liner and a spunbond-meltblown-spunbond barrier wrap material. The simulated spunbond liner was located on top of the surge layer 30 and the simulated barrier wrap material was located between the surge layer 30 and the absorbent pad 10.

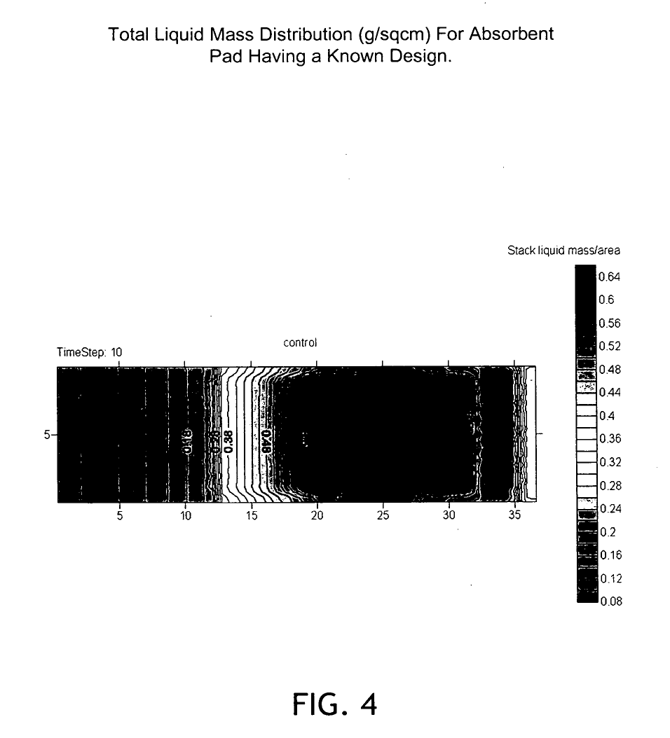

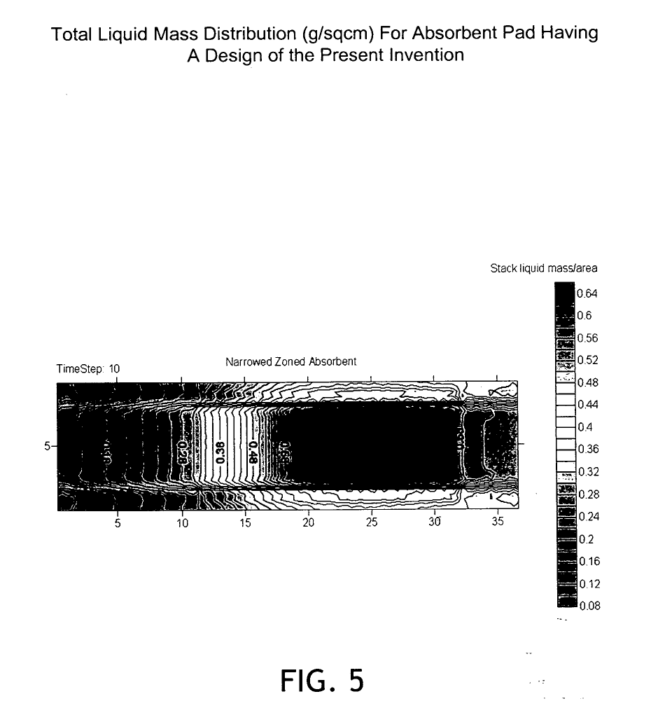

FIG. 4 representatively shows the total liquid mass distribution (g/cm²) for the absorbent pad 10 having a “known” design (e.g. the design shown in FIG. 1) after completion of the simulation events. FIG. 5 representatively shows the total liquid mass distribution (g/cm²) for the absorbent pad 10 having a design of the present invention (e.g. the design shown in FIG. 2) after completion of the same sequence of simulation events. Despite being loaded with the same mass of fluid (140 g), the distribution of liquid in the two absorbent pad designs is very different. As shown in FIG. 4, the “known” absorbent pad design loaded in such a way that more of the fluid remained in the region of the absorbent pad closest to the insult zone and the fluid spread over a greater portion of the width in the lateral direction of the absorbent pad. As shown in FIG. 5, the absorbent pad design of the invention loaded in such a way that the fluid distributed over a greater portion of the length of the absorbent pad and over less of the width. The difference in the longitudinal distribution of fluid is representatively illustrated by comparing the location of the 0.18 g/cm² contour in FIG. 4 versus FIG. 5. The 0.18 g/cm² contour in FIG. 5 is further away from the center of the absorbent pad than in FIG. 4. Additionally, due to the geometry of the absorbent pads in the simulations, the distribution of fluid in FIG. 5 represents fluid transport in an upward direction relative to the location of the insult zone. The distribution distances and intake times for the absorbent pad designs represented in FIG. 4 and FIG. 5 are quantitatively compared in Table 1. below.

TABLE 1

| | “Known” Absorbent | Absorbent Pad Design |
|---|---|---|
| | Pad Design | of the Invention |
| Distance Traveled by Fluid | 9.71 cm | 12.64 cm |
| Toward Front Edge of | | |
| Absorbent Pad | | |
| Distance Traveled by Fluid | 13.69 cm | 20.24 cm |
| Toward Back Edge of | | |
| Absorbent Pad | | |
| Intake Time for First Insult | 12.1 sec | 14.8 sec |
| Intake Time for Second | 25.9 sec | 32.7 sec |
| Insult | | |

The values in Table 1. compare the front and back liquid distributions for the absorbent pad designs shown in FIG. 4 and FIG. 5 by comparing the locations of the 0.53 g/cm² (contour line toward the front edge of the simulated absorbent pad; that is, toward the right edge of the simulated absorbent pad shown in FIG. 4 and FIG. 5) and 0.16 g/cm² (contour line toward the back edge of the simulated absorbent pad; that is toward the left edge of the simulated absorbent pad shown in FIG. 4 and FIG. 5) contours. While the 0.53 g/cm² and 0.16 g/cm² contour lines are not specifically labeled in FIG. 4 and FIG. 5, they may be located between the contour lines that are labeled. As described above, the liquid distribution in the longitudinal direction is more extensive and the liquid spreads less toward the longitudinal edges of the absorbent pad for the absorbent pad design of the invention. It is expected that the liquid distribution achieved by the absorbent pad design of the invention would result in less leakage because gasket function around the leg openings would be better maintained as a result of reduced thickness across the width of the absorbent pad. While the absorbent pad design of the invention has slightly longer intake times after both the first and second insults, the slightly increased times would not be expected to detrimentally affect performance of the absorbent pad. Based on the results of the simulations, it would be expected that an absorbent pad having a reduced width of the narrow zone area of absorbent material along with an increased thickness of the absorbent material (in order to maintain capacity) would further improve the final liquid distribution without significant impact on the intake time.

The narrow zone area of increased thickness of absorbent material may have a thickness that is up to about 1 cm greater than the thickness of absorbent material in the non-zone area of the absorbent pad. The density of absorbent material in the narrow zone area may be the same as the density of absorbent material in the non-zone area of the absorbent pad or it may have a different density. For example, it may be desirable to construct the narrow zone area to have a lower density than the non-zone area of the absorbent pad, but to form it from smaller diameter, high wetting fibers in order to maximize diffusivity. The properties of the narrow zone area may vary along the length and the width of the absorbent pad. For example, the density could gradually increase along the length of the narrow zone area between the insult zone and the lateral edges of the absorbent pad. The narrow zone area for absorbent systems of the invention may be formed by using zoning screens. Zoning screens may used to form absorbent pads 10 in which the narrow zone area 500 is located both toward the inner surface 31 and the outer surface 33 of the disposable absorbent article 200 in which the absorbent pad 10 is used (see FIGS. 13 and 14). Alternatively, the narrow zone area 500 may be formed by densifying an initially uniform thickness absorbent pad 10 along the longitudinal sides resulting in a central zone of lower density and higher thickness of absorbent material. Additionally, the absorbent pad 10 may be constructed of two or more layers that are adjacent to each other. In such a configuration, the narrow zone area 500 may be formed of one layer of absorbent material and the non-zone area may be formed of a second layer of absorbent material. The two layers may be attached to each other by adhesive, ultrasonic, thermal or other bonding techniques. The thickness of the narrow zone area 500 is the sum of the thicknesses of the individual layers and therefore, is greater than the thickness of the non-zone area layer alone.

A second series of computer simulations using finite element analysis was run to further illustrate the performance benefits of absorbent system designs of the present invention. Each of the absorbent system designs in the second series had several features in common: (1) the shape of the absorbent pads 10 was rectangular (except for the shaped absorbent examples) and the dimensions of the absorbent pads 10 were 36 centimeters by 8 centimeters; (2) the absorbent materials used to form the absorbent pads had a density of 0.24 grams/cubic centimeters, included 60% pulp fiber and 40% superabsorbent and had a contact angle of 35 degrees; (3) each absorbent system included a 0.04 centimeter barrier layer around the absorbent pad 10; (4) each absorbent system included a surge layer 30 having a density of 0.028 grams/cubic centimeters, except as will be noted; and (5) the absorbent systems did not include a liner. In the second series of simulations, the insult area was the same for each absorbent system and the simulations were run as if the user of the absorbent systems was in a standing position. For the examples having a narrow zone area of increased thickness of absorbent materials, the narrow zone area had a length in the longitudinal direction of the absorbent system equal to the length of the absorbent pad 10. The sequence of insult events for the second series of simulations included the following: (1) a 70 gram liquid insult; (2) a 120 second hold time (in which no liquid is allowed to enter the absorbent system, however liquid may re-distribute itself within the absorbent system); (3) a second, 70 gram liquid insult; (4) a second, 120 second hold time; and (5) a third, 70 gram liquid insult. The simulations included predictions of intake time and liquid distribution after completion of all of the simulation events. The results of the simulations are provided in Table 2. below.

TABLE 2

| | | Absorbent | Surge Density | Surge Thickness | First Intake | Second Intake | Third Intake |
|---|---|---|---|---|---|---|---|
| Code Name: | Code ID | Zone Ratio | (g/cm³⁾ | (cm) | Time (sec) | Time (sec) | Time (sec) |
| Conv. Zone StdS | 1 | 2.06 | 0.028 | 0.3 | 3.4 | 5.38 | 7.85 |
| NZA-50-StdSX | 2 | 3 | 0.028 | 0.4 | 3.61 | 5.38 | 7.46 |
| NZA-50-StdSX | 3 | 2 | 0.028 | 0.4 | 3.7 | 5.51 | 7.71 |
| Conv. Zone DStdS | 4 | 2.06 | 0.037 | 0.3 | 3.71 | 5.9 | 8.52 |
| NZA-50-StdS | 5 | 2 | 0.028 | 0.3 | 4.73 | 7.36 | 10.3 |
| NZA-75-StdS | 6 | 1.33 | 0.028 | 0.3 | 4.94 | 7.71 | 10.88 |
| FLAT-StdS | 7 | 1 | 0.028 | 0.3 | 5 | 7.76 | 10.97 |
| Shaped Absorbent | 8 | 2 | 0.028 | 0.4 | 5.32 | 6.62 | 9.42 |
| StdSX | | | | | | | |
| Shaped Absorbent | 9 | 2 | 0.028 | 0.3 | 6.48 | 8.61 | 12.23 |
| StdS | | | | | | | |
| Shaped NZA | 10 | 2 | 0.028 | 0.4 | 6.98 | 8.79 | 12.49 |
| StdSX | | | | | | | |

The Code Names in Table 2. represent the following absorbent systems:

“Conv. Zone StdS”=Conventional zoning of absorbent materials as shown in FIG. 1 with standard surge layer 30.

“NZA-50-StdSX”=Narrow zone area of absorbent materials wherein the narrow zone area has a width in the lateral direction of the absorbent system that is 50% of the width of the non-zone area of the absorbent pad 10 with standard surge layer 30, but the surge layer 30 having a greater thickness.

“Conv. Zone DStdS”=Conventional zoning of absorbent materials as shown in FIG. 1 with standard surge layer 30, but the surge layer 30 being of higher density.

“NZA-50-StdS”=Narrow zone area of absorbent materials wherein the narrow zone area has a width in the lateral direction of the absorbent system that is 50% of the width of the non-zone area of the absorbent pad 10 with standard surge layer 30.

“NZA-75-StdS”=Narrowed zone of absorbent materials wherein the narrowed zone has a width in the lateral direction of the absorbent system that is 75% of the width of the absorbent pad 10 with standard surge layer 30.

“FLAT-StdS”=Absorbent pad 10 that does not have any zoning of absorbent materials with standard surge layer 30.

“Shaped Absorbent StdSX”=Shaped absorbent pad 10 having a curved perimeter that would be located in the front waist region 23 of an absorbent article (as shown in FIG. 12) with standard surge layer 30, but the surge layer 30 having a greater thickness.

“Shaped Absorbent StdS”=Shaped absorbent pad 10 having a curved perimeter that would be located in the front waist region 23 of an absorbent article (as shown in FIG. 12) with standard surge layer 30.

“Shaped NZA StdSX”=Shaped absorbent pad 10 having a curved perimeter that would be located in the front waist region 23 of an absorbent article (as shown in FIG. 12) with a narrow zone area having a width in the lateral direction of the absorbent pad 10 that is 50% of the width of the non-zone area of the absorbent pad 10, also with standard surge layer 30, but the surge layer 30 having a greater thickness.

For the second series of computer simulations, the surge contact angle was 65 degrees for the rectangular absorbent pads 10 on all events in the simulations, including insults and holds. The surge contact angle was 65 degrees for the shaped absorbent pads 10 on the first insult and 50 degrees for the other events. Except for Code ID numbers 2, 6 and 7, each of the codes had a zoning ratio of approximately 2. Since density is uniform, the zoning ratio is the ratio of the thickness of the absorbent materials in a defined zone (e.g. narrow zone area) to the thickness of the absorbent materials in the remainder of the absorbent pad 10 (e.g. non-zone area).

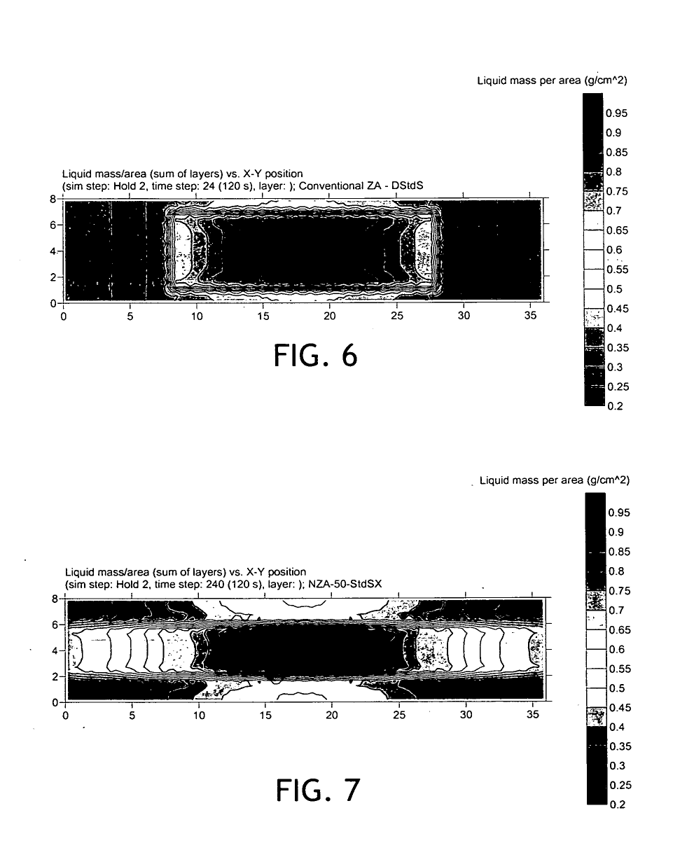

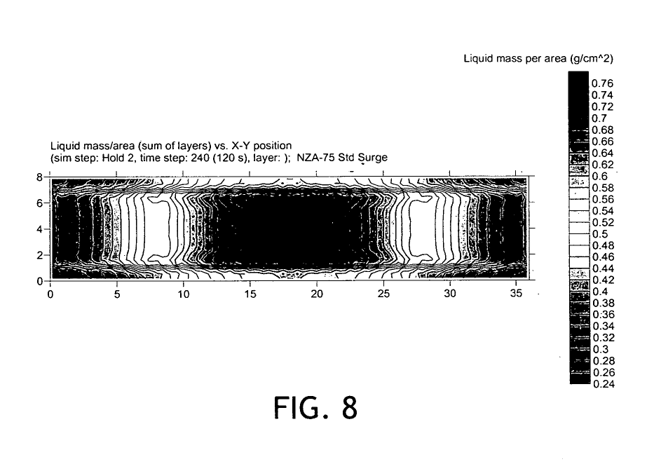

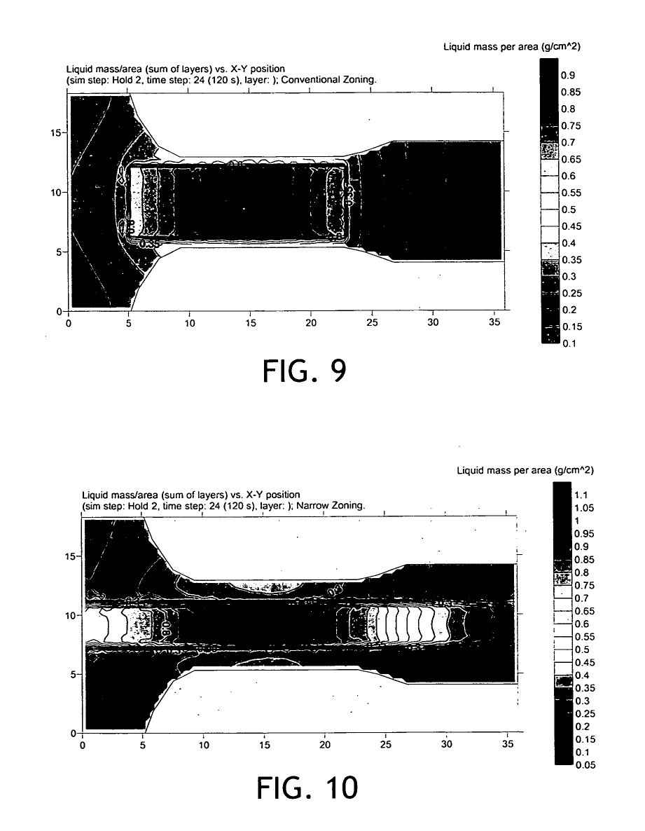

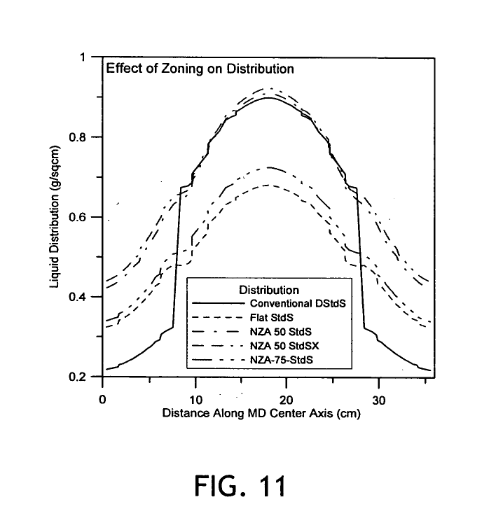

The results of the simulations provide a comparison between the performance of the “conventional” zone of absorbent material and the narrow zone area of absorbent material. One performance aspect of absorbent systems is their ability to intake fluids at a rapid rate. Comparing the First, Second and Third Intake Times for Code ID 1 to those for Code ID 5 and comparing the Intake Times for Code ID 8 to those for Code ID 10, the simulation results show that the “conventional” zones of absorbent material tend to intake multiple fluid insults in shorter amounts of time than the absorbent systems having narrow zone areas of absorbent material. However, comparing the Intake Times for Code ID 1 to those for Code IDs 2 and 3, the simulation results show that narrow zone areas of absorbent material combined with increased thickness surge layers 30 have comparable intake performance to absorbent systems having “conventional” zones of absorbent material. That being said, the intake performance of Code ID 5 (50% width narrow zone area of absorbent material) compared to Code ID 7 (a flat or uniform distribution of absorbent material) is better. Another performance aspect of absorbent systems is their ability to distribute fluid throughout the absorbent system and keep fluid away from the edges of the absorbent system to prevent or reduce leakage of fluid out of the absorbent system. The simulations also provide predicted fluid distributions for the different Code IDs; color representations of fluid distribution, including contour lines representing different magnitudes of liquid mass per area, are provided for Code ID 4 (FIG. 6); Code ID 3 (FIG. 7); Code ID 6 (FIG. 8); Code ID 9 (FIG. 9); and Code ID 10 (FIG. 10). FIG. 11 is a plot of Liquid Distribution (grams/square centimeters) along the longitudinal centerline of the absorbent pads 10 of Code ID numbers 3, 4, 5, 6 and 7 at the end of the second “hold” periods in the simulations. Looking at FIG. 11, the Code IDs having a narrow zone area of absorbent material that has a width in the lateral direction that is less than or equal to 62% of the width of the non-zone area of the absorbent pad and having a length substantially the same as the length of the absorbent pad perform better at distributing liquid to the far ends of the absorbent pads and at confining liquid along the longitudinal centerline.

Looking at the fluid distribution contour maps of FIGS. 6-10, Code ID 4 is represented in FIG. 6. With this “conventional” zone of absorbent material, there is very little distribution of fluid to the ends of the simulated absorbent pad and there is a heavy concentration of fluid near the longitudinal edges of the simulated absorbent pad, suggesting that leakage could occur. Code ID 6 is represented in FIG. 8 and simulates the performance of a zone of absorbent material that is approximately 75% of the width of the simulated absorbent pad. The fluid distribution for Code ID 6 is more spread out toward the ends of the simulated absorbent pad than it was for Code ID 4, but there is still a heavy concentration of fluid near the longitudinal edges. Code ID 3 is shown in FIG. 7 and represents an absorbent system of the invention. With this example, the fluid distribution extends to both ends of the simulated absorbent pad and there is a lighter concentration of fluid near the longitudinal edges. FIG. 9 shows the fluid distribution contour map for Code ID 9, “conventional” zoning of absorbent material within a shaped (rather than rectangular) absorbent pad. As with Code ID 4 and Code ID 6, the fluid is concentrated in the center of the pad and there is minimal distribution to the ends of the simulated absorbent pad. Additionally, there is a heavy concentration of fluid near the longitudinal edges. FIG. 10 shows the fluid distribution contour map for Code ID 10, an absorbent system of the invention. Code ID 10 includes a narrow zone area of absorbent material having a width that is approximately 50% of the width of the non-zone area of the simulated, shaped absorbent pad in a crotch region. As with Code ID 3, the fluid distribution extends to both ends of the absorbent pad and there is a lighter concentration of fluid near the longitudinal edges.

In conclusion, computer simulations of absorbent systems that are exposed to multiple insults show that absorbent pads that include a narrow zone area of additional thickness of absorbent material that extends approximately the full length of the absorbent pad have better fluid distribution than absorbent pads with “conventional” zoning. While absorbent pads having “conventional” zoning of absorbent material may have better intake times, the absorbent systems of the invention can achieve comparable intake performance through simple modifications such as changing the thickness of the surge material that is used.

While the absorbent systems of the invention have been described in detail with respect to specific aspects thereof, it will be appreciated that those skilled in the art, upon attaining an understanding of the foregoing, may readily conceive of alterations to, variations of and equivalents to these systems. Accordingly, the scope of the present invention should be assessed as that of the appended claims and any equivalents thereto.

We claim:

1. A disposable absorbent article comprising:
an absorbent system, wherein the absorbent system includes an absorbent pad formed of absorbent materials and having a lateral direction and a longitudinal direction, wherein the absorbent pad has a narrow zone area of increased thickness of absorbent materials, wherein the narrow zone area has a width in the lateral direction of the absorbent pad that is less than or equal to 62% and greater than or equal to 20% of the width of a non-zone area of the absorbent pad and the narrow zone area has a length in the longitudinal direction of the absorbent pad that is greater than or equal to 80% of the length of the absorbent pad.

2. The disposable absorbent article of claim 1, wherein the width of the narrow zone area is less than or equal to 55% of the width of the non-zone area.

3. The disposable absorbent article of claim 1, wherein the width of the narrow zone area is less than or equal to 50% of the width of the non-zone area.

4. The disposable absorbent article of claim 1 wherein the width of the narrow zone area is less than or equal to 45% of the width of the non-zone area.

5. The disposable absorbent article of claim 1 wherein the length of the narrow zone area is greater than or equal to 90% of the length of the absorbent pad.

6. The disposable absorbent article of claim 1 wherein the length of the narrow zone area is greater than or equal to 95% of the length of the absorbent pad.

7. The disposable absorbent article of claim 1, wherein the thickness of the narrow zone area is 1.2 to 5 times a thickness of the non-zone area.

8. The disposable absorbent article of claim 1, wherein the thickness of the narrow zone area is at least 0.1 centimeters greater than a thickness of the non-zone area.

9. The disposable absorbent article of claim 1, wherein the thickness of the narrow zone area is at least 0.4 centimeters greater than a thickness of the non-zone area.

10. The disposable absorbent article of claim 1, wherein a density of absorbent materials in the narrow zone area is the same as a density of absorbent materials in the non-zone area.

11. The disposable absorbent article of claim 1, wherein a density of absorbent materials in the narrow zone area is less than a density of absorbent materials in the non-zone area.

12. The disposable absorbent article of claim 1, further comprising a surge layer.

13. The disposable absorbent article of claim 1, wherein the narrow zone area and the non-zone area of the absorbent pad are a continuous structure.

14. The disposable absorbent article of claim 1, wherein the narrow zone area and the non-zone area of the absorbent pad are separate, but adjacent structures.
